(19) United States
(12) Patent Application Publication
(10) Pub. No.: US 2020/0406017 A1
(43) Pub. Date: Dec. 31, 2020

(54) TECHNIQUE AND METHOD TO LOCALLY DELIVER OBJECTS INTO BONE

(71) Applicant: Revenio Research Oy, Vantaa (FI)

(72) Inventors: Petro MOILANEN, Säynätsalo (FI); Heikki NIEMINEN, Helsinki (FI); Ari SALMI, Helsinki (FI); Edward HAEGGSTRÖM, Helsinki (FI); Alejandro GARCIA PEREZ, Helsinki (FI)

(73) Assignee: Revenio Research Oy, Vantaa (FI)

(21) Appl. No.: 17/017,339

(22) Filed: Sep. 10, 2020

Related U.S. Application Data
(63) Continuation-in-part of application No. 15/917,044, filed on Mar. 9, 2018, now abandoned.
Application No. 15/917,044 is a continuation of application No. PCT/FI2015/050589, filed on Sep. 9, 2015.

(51) Int. Cl. A61M 37/00

(52) U.S. Cl. CPC A61M 37/0092; A61B 8/0875; A61M 2205/0294; A61M 37/00

(57) ABSTRACT

An object delivery arrangement is disclosed for delivering objects into bone. The arrangement is configured for generating localized mechanical waves into a tissue, for performing localized deposition of the objects near bone, and for exposing the objects and the bone to said mechanical waves to obtain deposition of the objects into the bone.

### RELATED APPLICATIONS

This application is a continuation-in-part of U.S. application Ser. No. 15/917,044, filed on Mar. 9, 2018, which in turn is a continuation application of PCT/FI2015/050589, filed on Sep. 9, 2015, designating the U.S., the entire content of each of which is hereby incorporated by reference in its entirety.

### FIELD

The present disclosure relates to bone healthcare and health management. Exemplary embodiments deal with detection of a weak bone and healing of the weak or fractured bone in vivo.

### BACKGROUND INFORMATION

Bone diseases are disorders in remodeling of bone tissue. As a result, bones can become mechanically weak. Reduction of bone mineral density (BMD) is a natural process related to aging after the age of 20. However, some bone diseases, such as osteoporosis, can cause excessive loss of BMD. Deficiencies in nutrient intake (e.g., calcium and vitamin D and C), hormonal imbalance and cell abnormalities can also cause bone disorders.

Bone fractures are labelled low-impact fractures and high-impact fractures. The low-impact (or fragility) fractures are predominantly caused by deteriorated bone strength, which results from aging or bone disease, and can occur due to a mechanical impact following for example, slipping or falling. High-impact (or traumatic) fractures require excessive stress caused by traumatic accidents and can occur in healthy bone. Bone is considered weak when the risk for fragility fractures is increased. Bone strength/fragility is described in detail in Consensus development conference: diagnosis, prophylaxis and treatment of osteoporosis, Am J Med 94 (1993) 646-650.

There is a need for methods to detect and heal weak bones, preferably before fractures occur.

Localized inference of bone quality techniques are being developed by several research groups. To this end, quantitative ultrasound (QUS) is one of the most promising approaches. Yet, ultrasonic detection of clinically relevant fracture sites such as the hip and vertebrae is challenging and requires further development.

Weak bone is often treated by systemic delivery of drug and growth factors. Such drugs and drug-like factors are absorbed throughout the body. Therefore, high doses may be required to gain sufficient therapeutic effects in the bone. However, the drug, especially at high drug doses, may cause side effects outside fracture sites, some of which may be severe.

Tissue treatment based on localized delivery and release of drugs has been reported for soft tissue sites. For example, a recent report details ultrasound-aided delivery and release in articular cartilage (Nieminen et al., Ultrasound Med Biol 41(8):2259-2268, 2015) and subchondral bone through articular cartilage (Nieminen et al., Ultrasonics Symposium (IUS), 2012 IEEE International, pages 1869-1872). For bone metastases, there are reports on localized ultrasound-aided release of drugs, first transported into the vicinity of the therapy site by blood circulation (Staruch et al., Radiology 263(1):117-127, 2012). However, there is no known method to do simultaneous release and deposition. Moreover, there is no known methodology that would permit construction of a hand-held device for detection of weak bone (site with fracture risk) followed by instant localized treatment.

U.S. Pat. No. 6,231,528 B1 discloses an in vivo technology for using ultrasound in conjunction with a biomedical compound or bone growth factor to induce healing, growth and ingrowth responses in bone. To this end, non-invasively applied ultrasonic stimulus is operative to transport the bone growth factor from the external surface of the soft tissue to the bone and to synergistically enhance the interaction between the bone growth factor and the bone. This technology does not involve deposition of the ultrasonically transported objects and describes the use of ultrasound for delivery only in the context of an extracorporeal ultrasound transducer, an ultrasound pulser, biomedical compounds and bone growth factors. In addition, the technology does not incorporate focused ultrasonic waves which are vital for highly localized treatment.

### SUMMARY

A kit is disclosed, comprising: an object delivery arrangement for delivering objects into bone; and retention means configured to counteract passive diffusion out from a target and formed by one of: a covering layer having a lower perfusion coefficient than embracing tissues; an active object having a size sufficient to prevent passive diffusion out of a target in a bone; a substance that expands and covers a target site in the bone; and an ultrasound, photo-acoustics or plasma source configured to generate localized mechanical waves for maintaining a substance and for subsequently depositing the substance; wherein said arrangement is configured to: perform localized deposition of objects near a bone; expose objects and a bone to localized mechanical waves to force objects into a bone; and perform retention of deposited objects in a bone by using said retention means, so as to prevent deposited objects from escaping a target site in that bone.

### BRIEF DESCRIPTION OF THE DRAWINGS

Other features and advantages disclosed herein will become more apparent from the following detailed description of exemplary embodiments, when read in conjunction with the accompanying drawings wherein the elements are represented by like reference numerals.

FIG. 1 shows exemplary embodiments of the present disclosure.

FIG. 2 shows alternate exemplary embodiments of the present disclosure.

FIGS. 3(a) and 3(b) show exemplary preliminary results.

### DETAILED DESCRIPTION

An improved technology is disclosed for transport and deposition of objects into bone for effective and controllable localized management of bone health. This can, for example, be achieved by an object delivery arrangement for delivering objects into bone. The arrangement can include for generating localized mechanical waves into a tissue, means for performing localized deposition of objects near bone, and means for exposing the objects and the bone to the mechanical waves to obtain deposition of the objects into the bone.

An object delivery method is disclosed for delivering objects into bone. In the method localized mechanical waves can be generated into a tissue, localized deposition of the objects near bone is performed, and the objects and the bone are exposed to the mechanical waves to obtain deposition of the objects into the bone.

Exemplary embodiments are based on generation of localized mechanical waves into a tissue, and localized deposition of the objects near bone, and deposition of the objects to the bone by the effect of the mechanical waves.

The direction of transportation and deposition of objects into bone tissue is not limited to transport and deposition from bone periosteal (i.e., outer) surface into bone tissue. The transportation and deposition of objects can also be achieved from any surface of a cavity (e.g., endosteal surface) or pore into bone tissue.

An exemplary benefit of embodiments disclosed herein is that the proposed conjunction of means permits an enhanced therapeutic power and advanced management of the therapeutic effect compared to known treatments.

In an exemplary object delivery arrangement for delivering objects into bone as disclosed, the delivery object is, for example, a drug molecule or molecules for osteoporosis treatment. The arrangement can include means for generating localized mechanical waves into a tissue. The means are for example at least one of sound emitter 108, mechanical wave emitter 113, energy conductor 109, sound source 111, and waveguide 112. The means 108, 111 are for example an ultrasound transducer or an ultrasound source.

Sound emitter refers to an object emitting sound, e.g. an end of a waveguide. Sound source refers to an object that creates sound, e.g. a piezoelectric transducer. Mechanical wave emitter is an ultrasonic transducer or ultrasonic source 108 in direct contact with a tissue, or the head 113 of a mechanical waveguide 112 in direct contact with a tissue. The opposite end of the mechanical waveguide is coupled to an sound source 111. There is no fundamental difference between the source devices 108 and 111. Source translates electromagnetic energy into mechanical energy and emitter delivers the mechanical energy into tissue.

The arrangement can include means for performing localized deposition of the objects in a reservoir 103 contained within a material boundary 104 near bone interface 107, and means for exposing the objects and the bone to the mechanical waves, obtaining deposition 110 of the objects to the bone. The means for performing localized deposition are for example at least one of hollow structures: catheter 101, needle 201, cutting edge 102 of the needle or catheter, reservoir 200 and syringe 203. The means for exposing are for example at least one of the elements represented by reference signs 108, 109, 111, 112 and 113. The means 108, 109, 111, 112, 113 can penetrate skin 105 or tissues 106.

An exemplary arrangement according to the present disclosure can include means for transporting the objects to the near bone interface 107 in order to obtain deposition of the objects 110 to the bone. The means for transporting are for example at least one of means according to reference signs 101 (catheter), 102 (cutting edge), 103 (a reservoir), 108 and 109. In an exemplary embodiment the location of weak bone is detected by quantitative ultrasound (QUS) or ultrasound imaging.

Clinical utilization of QUS has been discussed, e.g., in Krieg et al., Quantitative Ultrasound in the Management of Osteoporosis: The 2007 ISCD Official Positions, Journal of Clinical Densitometry 11(1): 163-187, 2008. Detailed examples on suitable QUS techniques have been described, e.g., in WO2013064740 to Moilanen et al., A skeletal method and arrangement utilizing electromagnetic waves, WO2009109695 to Pukki, A method and a device for measuring density of a bone, WO03045251 to Moilanen et al., A method for the non-invasive assessment of bones.

After defining the weak bone, the deposition of objects into the weak bone can be achieved with mechanical waves generated with the same or a different ultrasound system than used for QUS or ultrasound imaging.

An arrangement according to the present disclosure can include means, such as at least one of means according to reference signs 108, 113, 109, 111, 112 and ultrasound generators 204a-e, for generating localized mechanical waves into the tissue to perform the localization on the basis of at least one of high-intensity focused ultrasound (HIFU) or ultrasound generator 204c, topological guides for ultrasound or waveguide 112 and electromagnetic steering of the objects to improve focusing of the diffusion. See, e.g., US2019321013 (A1) to Nieminen et al., Method and apparatus for extracting and delivery of entities, WO2017021585 to Garcia-Perez et al., Device for localized delivery and extraction of material, Nieminen et al., Ultrasonic transport of particles into articular cartilage and subchondral bone, 2012 IEEE International Ultrasonics Symposium, DOI: 10.1109/ULTSYM.2012.0469.

According to exemplary embodiments, it is essential for localized deposition to localize the driving mechanical (or sound) wave field inside the tissue, at for example, a preferred point at or near the bone (e.g. a weak part of the bone). Localization of the driving mechanical wave field can be realized either by means of high-intensity focused ultrasound (HIFU) or topological ultrasound (waveguides or high-order topologies such as fractal structures).

Localization can also be realized by means of a counter electrode, or electromagnetic fields that steer the field or objects, to improve focusing of the diffusion. Waveguides for guiding sound have been well known in the literature, e.g. https://www.sciencedirect.com/science/article/pii/0041624X63900611. High intensity focused ultrasound is per definition focused to a spot (as is stated in the name “focused ultrasound”). Electromagnetic steering can use principles well known in the literature for guiding e.g. nanoswimmers (https://www.tandfonline.com/doi/full/10.1080/21553769.2014.962103).

In an exemplary embodiment according to the present disclosure the arrangement can include means, such as at least one of means according to reference signs 108, 113, 109, 111, 112 and 204a-e, for generating localized mechanical waves into a tissue on the basis of time reversal ultrasound performing adaptive focusing. Time reversal ultrasound permits adaptive focusing through an inhomogeneous medium, such as soft tissue or trabecular bone.

Time reversal focusing works by sending a pulse into the tissue, listening to the response and then re-transmitting the received wave time reversed. This focuses the energy to the scatterers. More detailed explanation can be found e.g., in Mathias Fink et al 2000 Rep. Prog. Phys. 63, pp 1933-1995; Mathias Fink, Time-Reversed Acoustics, Scientific American, November 1999, pp. 91-97; and Time Reversal of Ultrasonic Fields-Part I: Basic Principles, IEEE Transactions on Ultrasonics, Ferroelectrics, and Frequency Control, 1992, Vol. 39, No. 5, pp. 555-566. Time reversal has been a wellknown technique, also for tissue based focusing (e.g., Thomas et al., Ultrasonic beam focusing through tissue inhomogeneities with a time reversal mirror: application to transskull therapy, IEEE Transactions on Ultrasonics, Ferroelectrics, and Frequency Control, Volume: 43, Issue: 6, November 1996, pp. 1122-1129, at https://ieeexplore.ieee.org/absract/document/542055).

The arrangement can also include means, such as at least one of means according to reference signs 108, 113, 109, 111, 112, 200, 201, 203 and 204a-e, for performing localized deposition of the objects near bone based on photo-acoustic transformation in order to generate mechanical waves near objects. Photo-acoustic transformation also permits introduction of the sound source inside the tissue.

In this approach the tissue is irradiated by electromagnetic waves (e.g., laser pulse), which penetrate and absorb into the tissue. The absorption causes localized thermal expansion, which results in emission of mechanical waves (e.g., a sound field) at the spot of thermal expansion. The resulting sound field is tunable by parameters of the optical beam (e.g., wavelength, pulse duration, geometric size and shape of the optical beam, number of illuminated spots and/or temporal phasing of an onset of illumination of the different spots). These parameters affect the penetration depth, absorption and scattering in the tissue and determine the emitted sound field. The energy of the electromagnetic beam can be absorbed in any parts of the tissue or in the objects that are being deposited or a combination of thereof. For example, the optical absorption coefficients characteristic to different layers of the soft tissue and bone are functions of the optical wavelength. Thereby, tuning of the optical wavelength permits for example maximization of an absorption ratio between the bone and soft tissue and can result in localization of the sound source at or near the bone.

The suitable electromagnetic waves includes, e.g., those described in WO2013064740 to Moilanen et al., A skeletal method and arrangement utilizing electromagnetic waves. The localization can be also obtained by using a point source (e.g. 108, 113, 204a-b), independent of the technique of implementation. It has been well known that vibration can enhance drug (i.e., substance) delivery into tissue, e.g. WO2014105754A1, https://www.ncbi.nlm.nih.gov/pubmed/23121385.

In another exemplary embodiment according to the present disclosure, the arrangement includes means for selecting the objects from a reservoir of objects and forcing the objects into the bone. Objects (e.g., molecules) are selected from the reservoir of objects (e.g., solution) and are forced into the bone. The selection of the desired object can be carried out, e.g., by selecting a suitable frequency for mechanical waves, which interact with the objects that are close to the size of the wavelength of the waves.

The arrangement can include means, such as at least one of means according to reference signs 108, 113, 109, 111, 112, 200, 201, 203 and 204a-e, for performing retention of objects in a reservoir 103, 110 by depositing in addition to the objects a covering layer having a lower perfusion coefficient than the embracing tissues. The purpose of retention is to prevent the deposed objects from escaping the target (bone). Origami metamaterials (e.g. Silverberg et al., https://science.sciencemag.org/content/345/6197/647) can be changed using, e.g., external energy input at a desired time. This is also published, e.g., in Hauser et al., https://onlinelibraty.wiley.com/doi/full/10.1002/anie.201412160. In US 20150086602A1 it has been presented that layer methods can be used for coating bone allografts with periosteum-mimetic tissue engineering scaffolds to form artificial periosteum, i.e. a layer that isolates the bone from the surroundings.

Self-assembly type of technology can be utilized to drive into a bone some compound to form a fiber mesh around the bone. Covering layers and substances, and active objects have been well known in the literature, see, e.g., self-assembly of materials that can be deposited (https://pubs.acs.org/doi/abs/10.1021/ja904411z) or materials that have been used to cover bone allografts, e.g. US 2015/0086602.

Exemplary substrates are drug molecules, prodrug molecules, drug candidate molecules, nanoparticles, gold particles, such as gold nanoparticles, cells, viruses, bisphosphonates, steroids, proteoglycan, collagen, and growth factors. According to another embodiment, the substrate is selected from drug molecules, gold particles, viruses and cells.

Biologically active materials that may be of interest to the technology include analgesics, antagonists, anti-inflammatory agents, anthelmintics, antianginal agents, antiarrhythmic agents, antibiotics (including penicillins), anticholesterols, anticoagulants, anticonvulsants, antidepressants, antidiabetic agents, antiepileptics, antigonadotropins, antihistamines, antihypertensive agents, antimuscarinic agents, antimycobacterial agents, antineoplastic agents, antipsychotic agents, immunosuppressants, antithyroid agents, antiviral agents, antifungal agents, anxiolytic sedatives (hypnotics and neuroleptics), astringents, beta-adrenoceptor blocking agents, blood products and substitutes, anti-cancer agents, card iacinotropic agents, contrast media, corticosterioids, cough suppressants (expectorants and mucolytics), diuretics, dopaminergics (antiparkinsonian agents), haemostatics, immunosuppressive and immunoactive agents, lipid regulating agents, muscle relaxants, parasympathomimetics, parathyroid calcitonin and biphosphonates, prostaglandins, radiopharmaceuticals, sex hormones (including steroids), anti-allergic agents, stimulants and anorexics, sympathomimetics, thyroid agents, vasidilators, neuron blocking agents, anticholinergic and cholinomimetic agents, antimuscarinic and muscarinic agents, vitamins, and xanthines.

Exemplary medicaments suitable for the present technology are entacapone, esomeprazole, atorvastatin, rabeprazole, piroxicam and olanzapine. An exemplary medicament is piroxicam (4-hydroxy-2-methyl-N-(2-pyridinyl)-2H-1,2-benzothiazine-3-carboxamide 1,1-dioxide). Retention is realized by deposing a covering layer that has much lower perfusion coefficient compared to those of the embracing tissues. This can also be realized by depositing a substance that expands and covers the target (i.e., the deposited object). The substance that expands and covers the target may include, e.g., viscoelastic gels that change viscosity as a function of temperature—the localized temperature can be controlled e.g. by localized infrared radiation or localization of mechanical energy.

An alternative embodiment to this approach is using an active object, which is too large for passive diffusion, but can be actively deposited using the method(s) described herein. The active object may include, e.g., origami metamaterials or viscoelastic objects that change their stiffness as a function of temperature. The large size which is larger than the pores of the material in question can then prevent passive diffusion out of the target.

Retention can also be controlled by subsequent sonication: a first application of mechanical waves deposits a substance into the target, followed by several applications of mechanical waves that maintain the substance in the target (counteract the passive diffusion out from the target).

In an exemplary embodiment according to the present disclosure the arrangement can include means, such as at least one of means according to reference signs 101, 102, 103, 108, 113, 109, 111, 112, 200, 201, 203 and 204a-e, for activating objects selectively at different time points. Objects that are inactive in the tissue are first driven in, to form a reservoir 103, boundary 104, reservoir 110 of the objects in the tissue. After this, the objects are collectively or selectively activated such as by means of mechanical waves, electromagnetic waves or temperature. Selective activation permits activation at different time points; for example, one ingredient of the objects can be activated directly after drive in and another ingredient can be activated later. This can be considered to be for example catalyzation.

Examples of inactive objects or another ingredients can be found in, e.g., lipid-coated bubbles that are already in use. In these, the bubble's Minnaert resonance is matched with the frequency of an externally applied ultrasound field, which explodes the bubbles, e.g., https://www.ncbi.nlm.nih.gov/pmc/articles/PMC2807701/. This can be done selectively by adjusting the frequency and driving in bubbles of varying sizes.

In an alternative embodiment, the different drugs are encapsulated in or on a surface of for example, gas voids (with or without lipid shells or equivalent) of various sizes corresponding to various resonant frequencies. Self-assembly type of technology can be utilized to drive into a bone some compound to form a fiber mesh around the bone. Covering layers and substances, and active objects have been well known in the literature, see e.g. self-assembly of materials that can be deposited (https://pubs.acs.org/doi/abs/10.1021/ja904411z) or materials that have been used to cover bone allografts, e.g. US 2015/0086602. Exemplary substrates are drug molecules, prodrug molecules, drug candidate molecules, nanoparticles, gold particles, such as gold nanoparticles, cells, viruses, bisphosphonates, steroids, proteoglycan, collagen, and growth factors. According to another embodiment, the substrate is selected from drug molecules, gold particles, viruses and cells.

Biologically active materials that may be of interest to the technology include analgesics, antagonists, anti-inflammatory agents, anthelmintics, antianginal agents, antiarrhythmic agents, antibiotics (including penicillins), anticholesterols, anticoagulants, anticonvulsants, antidepressants, antidiabetic agents, antiepileptics, a ntigonadotropins, antihistamines, antihypertensive agents, antimuscarinic agents, antimycobacterial agents, antineoplastic agents, antipsychotic agents, immunosuppressants, antithyroid agents, antiviral agents, antifungal agents, anxiolytic sedatives (hypnotics and neuroleptics), astringents, beta-adrenoceptor blocking agents, blood products and substitutes, anti-cancer agents, card iacinotropic agents, contrast media, corticosterioids, cough suppressants (expectorants and mucolytics), diuretics, dopaminergics (antiparkinsonian agents), haemostatics, immunosuppressive and immunoactive agents, lipid regulating agents, muscle relaxants, parasympathomimetics, parathyroid calcitonin and biphosphonates, prostaglandins, radiopharmaceuticals, sex hormones (including steroids), anti-allergic agents, stimulants and anorexics, sympathomimetics, thyroid agents, vasidilators, neuron blocking agents, anticholinergic and cholinomimetic agents, antimuscarinic and muscarinic agents, vitamins, and xanthines.

Exemplary medicaments suitable for the present technology are entacapone, esomeprazole, atorvastatin, rabeprazole, piroxicam and olanzapine. An exemplary medicament is piroxicam (4-hydroxy-2-methyl-N-(2-pyridinyl)-2H-1,2-benzothiazine-3-carboxamide 1,1-dioxide).

After driving in, the encapsulated objects are released at desired moments by sonicating at the resonant frequency corresponding to the release of objects desired. Some examples are provided below.

| Drug | Action | Size | | Dose |
|---|---|---|---|---|
| Ibandronic | Slows bone | 270 | Da | 2.5 mg/d or 150 |
| acid (BPA) | resorption | | | mg/mo or 3 mg/3 |
| | | | | mo injection |
| Zoledronic | Slows bone | 272 | Da | 5 mg/a |
| acid | resorption | | | |
| Teriparatide | Speed up bone | 4 100 | Da | 20 μ/d injection |
| | regeneration | | | |
| Denosumab | Speed up bone | 145 000 | Da | 6 mg/6 mo injec- |
| | regeneration | | | tion |

| | | |
|---|---|---|
| TGF-beta | Bone generation | 25 000-50 000 Da |
| FGP-beta | Bone generation | 7 000-38 000 Da |
| BB1/biopharm | | |

The arrangement can also include means, such as at least one of means according to reference signs 108, 113, 109, 111, 112, 204a-e, for affecting tissue in the bone surface 107, tissue in the skin 105, tissue 106, tissue in the reservoir 110 and tissue 205 by mechanical vibrations. In assembly in situ or in vivo embodiments one, two or several components are driven in the tissue and then treated (shaken) by mechanical vibration. This shaking causes merging of the components to larger aggregates that cannot escape from the target tissue (e.g., bone) or whose escape rate is decreased.

In nanotechnology embodiments according to the present disclosure the arrangement according to the present disclosure can include nanostructure means to control diffusion and to amplify the diffusion. Nano-swimmers or functionalized nano-rods permit improved control of the diffusion and amplification of the diffusion. The same can also be accomplished for example by nano motors, which are controlled by at least one of external field, internal field, external power source and internal power source.

Nano-swimmers, functionalized nano-rods and nano motors have been well known in the literature. E.g., in Gao et al. has been described (p. 10487-10488): “An ultrasound (US) field was used to trigger the breakage of the outer shell of the LbL assembled polyelectrolyte multilayer microcapsules and release the encapsulated drugs.”

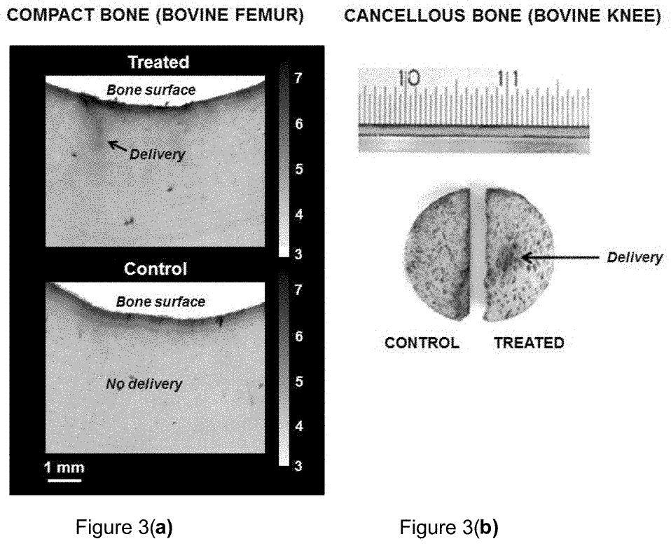

This is considered localized deposition of objects 103, assisted in this case by ultrasound (or mechanical waves). In a further step can be included localized delivery of the deposited objects from the bone surface into the bone by mechanical waves (such as high-intensity focused ultrasound beam, HIU), according to experiment shown in FIGS. 3(a) and 3(b).

Self-assembly type of technology can be utilized to drive into a bone some compound to form a fiber mesh around the bone. Covering layers and substances, and active objects have been well known in the literature, see e.g. self-assembly of materials that can be deposited (https://pubs.acs.org/doi/abs/10.1021/ja904411z) or materials that have been used to cover bone allografts, e.g. US 2015/0086602. Exemplary substrates are drug molecules, prodrug molecules, drug candidate molecules, nanoparticles, gold particles, such as gold nanoparticles, cells, viruses, bisphosphonates, steroids, proteo-glycan, collagen, and growth factors. According to another embodiment, the substrate is selected from drug molecules, gold particles, viruses and cells.

Biologically active materials that may be of interest to the technology include anal-gesics, antagonists, anti-inflammatory agents, anthelmintics, antianginal agents, anti-arrhythmic agents, antibiotics (including penicillins), a nticholesterols, anticoagulants, anticonvulsants, antidepressants, antidiabetic agents, antiepileptics, antigonadotropins, antihistamines, antihypertensive agents, antimuscarinic agents, antimycobacterial agents, antineoplastic agents, antipsychotic agents, immunosuppressants, antithyroid agents, antiviral agents, antifungal agents, anxiolytic sedatives (hypnotics and neuro-leptics), astringents, beta-adrenoceptor blocking agents, blood products and substitutes, anti-cancer agents, card iacinotropic agents, contrast media, corticosterioids, cough suppressants (expectorants and mucolytics), diuretics, dopaminergics (antiparkinsonian agents), haemostatics, immunosuppressive and immunoactive agents, lipid regulating agents, muscle relaxants, parasympathomimetics, parathyroid calcitonin and biphosphonates, prostaglandins, radiopharmaceuticals, sex hormones (including steroids), anti-allergic agents, stimulants and anorexics, sympathomimetics, thyroid agents, vasidilators, neuron blocking agents, anticholinergic and cholinomimetic agents, antimuscarinic and muscarinic agents, vitamins, and xanthines.

Exemplary medicaments suitable for the present technology are entacapone, esomeprazole, atorvastatin, rabeprazole, piroxicam and olanzapine. An exemplary medicament is piroxicam (4-hydroxy-2-methyl-N-(2-pyridinyl)-2H-1,2-benzothiazine-3-carboxamide 1,1-dioxide).

In an exemplary embodiment according to the present disclosure the arrangement can include means (204a-e) for exposing the objects and the bone to the mechanical waves to deposit the objects to the bone utilizing at least one of blood circulation and the bone marrow cavity for the transportation of the objects. Alternatively, instead of driving from the periosteal side of the bone, the objects are driven in bone from the inside (e.g., endosteal side) (means 204b), utilizing blood circulation and/or the bone marrow cavity for the initial transport of the objects to the treatment site, for example, the fracture site, and then exploiting mechanical waves to deposit the objects into the bone.

In exemplary embodiments the arrangement according to the present disclosure can include multi-center-frequency means, such as at least one of means according to reference signs 108, 113, 109, 111, 112 and 204a-e, to generate mechanical waves of at least two different frequencies in order to improve transportation of the objects and deposition of the objects.

Generation of sound waves by at least at two distinct center-frequencies can enhance the drive in. For instance, a kilohertz frequency transducer (e.g., 204d) can be used to increase the permeability at the bone surface (e.g., periosteum or endosteum) and a megahertz frequency transducer (e.g., 204e) can be used to push the objects in.

In exemplary embodiments according to the present disclosure the means, such as at least one of means according to reference signs 108, 113, 109, 111, 112 and 204a-e, for generating localized mechanical waves into a tissue can include a plasma source. Alternatively, instead of using a known ultrasound or photo-acoustic approach, the driving pressure field can be generated by a plasma source. The plasma source can be realized for example, by a focused laser or a spark gap.

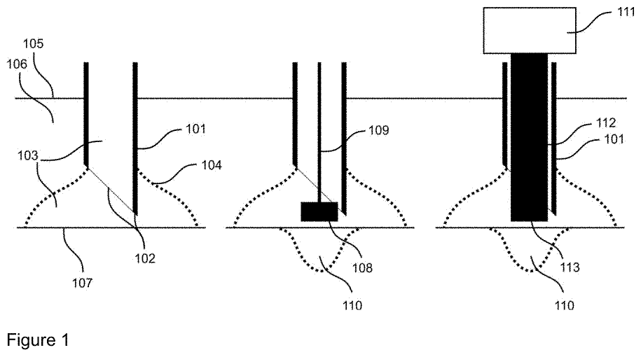

FIG. 1 depicts an exemplary embodiment of the disclosure. A catheter 101 featuring a cutting edge 102 perforates the skin and tissue. The catheter delivers the objects and it forms the reservoir 103 of the objects with boundary 104 near the bone surface 107. The sound source includes an energy conductor 109 and a sound emitter 108. The sound emitter 108 may be for example, a flat piezo, a focused piezo, spark, laser induced spark, EMUT (Energy Mode Ultrasound Transducer), CMUT (Capacitive micromachined ultrasonic transducers), PMUT (Piezoelectric Micromachined Ultrasonic Transducers) and equivalent. The mechanical wave generated by the sound emitter translates the objects into the reservoir 110 in the bone.

In another exemplary embodiment 111 is a sound source located outside the tissue and the mechanical wave is transmitted to the tissue and active ingredient via a waveguide 112. In an exemplary embodiment of the disclosure, for example, a 10-500 kHz mechanical wave is transmitted through at least one of waveguide 112, active ingredient in the reservoir 103, tissue 106 and sound emitter 108. This mechanical wave alters the permeability of the bone membrane. Another, for example, 0.5-50 MHz, mechanical wave is subsequently transmitted to the boundary. This mechanical wave deposits the active ingredient from the reservoir to the bone. For a person skilled in the art it is clear that objects respond to various frequencies that are close to their resonant frequencies, see e.g. Minnaert resonances of bubbles (Minnaert 1933). Thus, one can have a plethora of different sized objects, e.g. lipid bubbles containing active ingredients (e.g., https://www.ncbi.nlm.hih.gov/pmc/articles/PMC2727628/, https://ieeexplore.ieee.org/document/5935841) and then use the frequency to control which lipid bubble size is selected.

Deposition of objects through tissue by sound has been well known technology (‘sonophoresis’), see e.g. https://www.ncbi.nlm.nih.gov/pubmed/15019748, https://www.sciencedirect.com/science/article/pii/S0041624X13001947, https://onlinelibrary.wiley.com/doi/full/10.1211/jpp.61.06.0001).

According to an exemplary embodiment 111 is a light source 111 and a light wave is guided to a reservoir 103, boundary 104 and bone surface 107 through an optical fiber 112 or reflecting inner wall of a catheter 101. The light wave is absorbed by active ingredient in the reservoir 103 or bone surface 107 to generate light-induced sound waves for translating the active ingredient in the reservoir 103 into the bone through bone surface 107.

The object can be for example, molecules, drugs, vehicles carrying the object, imaging contrast agent, minerals or nanofibers. Biologically active materials that may be of interest include analgesics, antagonists, anti-inflammatory agents, anthelmintics, antianginal agents, antiarrhythmic agents, antibiotics (including penicillins), anticholesterols, anticoagulants, anticonvulsants, antidepressants, antidiabetic agents, antiepileptics, antigonadotropins, antihistamines, antihypertensive agents, antimuscarinic agents, antimycobacterial agents, antineoplastic agents, antipsychotic agents, immunosuppressants, antithyroid agents, antiviral agents, antifungal agents, anxiolytic sedatives (hypnotics and neuroleptics), astringents, beta-adrenoceptor blocking agents, blood products and substitutes, anti-cancer agents, cardiacinotropic agents, contrast media, corticosterioids, cough suppressants (expectorants and mucolytics), diuretics, dopaminergics (antiparkinsonian agents), haemostatics, immunosuppressive and immunoactive agents, lipid regulating agents, muscle relaxants, parasympathomimetics, parathyroid calcitonin and biphosphonates, prostaglandins, radiopharmaceuticals, sex hormones (including steroids), anti-allergic agents, stimulants and anorexics, sympathomimetics, thyroid agents, vasidilators, neuron blocking agents, anticholinergic and cholinomimetic agents, antimuscarinic and muscarinic agents, vitamins, and xanthines. Exemplary medicaments can be e.g. ibandronic acid, zolendronic acid, teriparatide, denosumab, TGF-beta, FGF-beta and BB1/biopharm.

According to an exemplary embodiment, the sound emitter 108 is a confocal transducer featuring two transducers of different center frequencies. According to an exemplary embodiment, the two frequencies generate a third frequency which acts as the wave translating the active ingredient. See, e.g., “Using Ultrasound Radiation Force for Noncontact Modal Testing of Hard-Drive Sus-pensions”, https://www.azooptics.com/Article.aspx?ArticleID=1599; Heath Martin et al., DualFrequency Piezoelectric Transducers for Contrast Enhanced Ultrasound Imaging, Sensors 2014, 14(11), 20825-20842; https://doi.org/10.3390/s141120825.

According to an exemplary embodiment, the wall of the catheter 101 or waveguide 112 acts as a “cold finger”, such that heat energy is absorbed from the tissues exposed to ultrasound induced heating.

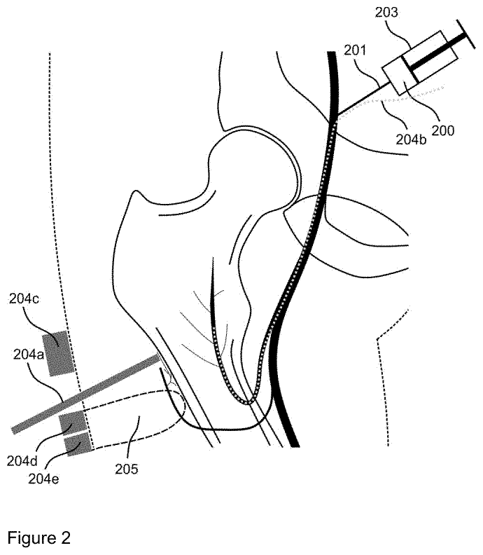

FIG. 2 depicts another means of translating objects, such as active ingredients, into bone. A syringe 203, loaded with the objects containing active ingredient in the reservoir 200, is connected with a needle 201 to a major artery which transports the objects with the blood flow to the treatment site. An ultrasound generator 204a, having at least one of 101, 103, 108, 111, 112, 113, 109 generates the ultrasound which locally translates the drug into the bone. In an alternative embodiment the ultrasound system 204b operates intravenously. In another alternative ultrasound system 204c, the ultrasound waves are focused through the skin and tissue to the reservoir 103, boundary 104, and bone surface 107 with an ultrasound generator 204c. In another alternative embodiment, a combination ultrasound system having two different transducers, one of which 204d translates the active ingredient through the tissue 205 and skin (such as sonophoresis), whereas the other one 204e translates the active ingredient into the bone.

FIGS. 3(a) and 3(b) show exemplary preliminary results on compact cortical and spongy cancellous bone. FIG. 3(a) Optical microscopy image of cortical bone into which has been delivered contrast agent (methylene blue; image on top) by using high-intensity focused ultrasound (HIU) (Parameters: sine burst frequency: 2.17 MHz; cycles per burst: 200, pulse-repetition frequency: 1000 Hz). The gray scale represents optical absorption. The ultrasound beam enhanced the delivery, as is indicated by an arrow. There is no similar effect seen in a control sample (image on bottom), extracted from the same piece of bone and treated consistently but without ultrasound. FIG. 3(b) Photograph of the result of a related experiment in cancellous bone (sine burst frequency: 2.17 MHz; cycles per burst: 100, pulse-repetition frequency: 600 Hz).

The experiments shown in FIGS. 3(a) and 3(b) do not include localized deposition, such as presented in schematic FIG. 1, but the bone samples are fully immersed into a liquid containing objects 103 (in this case contrast agent) and then HIU ultrasound beam was focused on the bone surface (interface) to enhance the delivery of contrast agent into the bone.

Localized delivery of objects into the bone includes transport and deposition according to the preferred or alternative exemplary embodiments of the disclosure as described in FIGS. 1 and 2. In one phase, one object or a group of objects is transported into the bone. In the second phase, a second object is transported into bone. The second object is delivered close to the pathways through which the first object has travelled to prevent washout of first object. The second object can alternatively selfassemble with itself or with the first object to create large-sized constructs (e.g., via mechanisms such as self-assembly) to slow down or prevent washout of the objects with therapeutic effect. The role of the second object can also be to catalyze the therapeutic effect of first object. The catalyzation can be achieved also by exposing at least one of the objects to mechanical or electromagnetic waves.

Self-assembly type of technology can be utilized to drive into a bone some compound to form a fiber mesh around the bone. Covering layers and substances, and active objects have been well known in the literature, see e.g. self-assembly of materials that can be deposited (https://pubs.acs.org/doi/abs/10.1021/ja904411z) or materials that have been used to cover bone allografts, e.g. US 2015/0086602. Exemplary substrates are drug molecules, prodrug molecules, drug candidate molecules, nanoparticles, gold particles, such as gold nanoparticles, cells, viruses, bisphosphonates, steroids, proteo-glycan, collagen, and growth factors. According to another embodiment, the substrate is selected from drug molecules, gold particles, viruses and cells.

Biologically active materials that may be of interest to the technology include anal-gesics, antagonists, anti-inflammatory agents, anthelmintics, antianginal agents, anti-arrhythmic agents, antibiotics (including penicillins), a nticholesterols, anticoagulants, anticonvulsants, antidepressants, antidiabetic agents, antiepileptics, antigonadotropins, antihistamines, antihypertensive agents, antimuscarinic agents, antimycobacterial agents, antineoplastic agents, antipsychotic agents, immunosuppressants, antithyroid agents, antiviral agents, antifungal agents, anxiolytic sedatives (hypnotics and neuro-leptics), astringents, beta-adrenoceptor blocking agents, blood products and substitutes, anti-cancer agents, card iacinotropic agents, contrast media, corticosterioids, cough suppressants (expectorants and mucolytics), diuretics, dopaminergics (antiparkinsonian agents), haemostatics, immunosuppressive and immunoactive agents, lipid regulating agents, muscle relaxants, parasympathomimetics, parathyroid calcitonin and biphosphonates, prostaglandins, radiopharmaceuticals, sex hormones (including steroids), anti-allergic agents, stimulants and anorexics, sympathomimetics, thyroid agents, vasidilators, neuron blocking agents, anticholinergic and cholinomimetic agents, antimuscarinic and muscarinic agents, vitamins, and xanthines.

Exemplary medicaments suitable for the present technology are entacapone, esomeprazole, atorvastatin, rabeprazole, piroxicam and olanzapine. An exemplary medicament is piroxicam (4-hydroxy-2-methyl-N-(2-pyridinyl)-2H-1,2-benzothiazine-3-carboxamide 1,1-dioxide).

Localized deposition is realized by employing one of the presented or different combinations of the presented techniques and methods.

Thus, it will be appreciated by those skilled in the art that the present invention can be embodied in other specific forms without departing from the spirit or essential characteristics thereof. The presently disclosed embodiments are therefore considered in all respects to be illustrative and not restricted. The scope of the invention is indicated by the appended claims rather than the foregoing description and all changes that come within the meaning and range and equivalence thereof are intended to be embraced therein.

1. A method of treating a bone in need thereof, comprising:
delivering a pharmaceutically effective amount of a pharmaceutical composition containing at least one object to a bone surface of a bone,
retaining the delivered at least one object at the bone surface, and
depositing the retained at least one object into the bone,
wherein the object contains a drug or a precursor thereof.

2. The method of claim 1, wherein the drug is a drug effective for osteoporosis treatment.

3. The method of claim 1, wherein the delivering is carried out by a catheter or syringe.

4. The method of claim 3, wherein the delivering is further carried out by a blood circulation or a bone marrow cavity.

5. The method of claim 3, wherein the delivering is carried out further by an ultrasound, photo-acoustic or plasma source.

6. The method of claim 1, wherein the retaining is carried out by providing:
a covering layer having a lower perfusion coefficient than tissues surrounding the delivered at least one object;
an active object having a size sufficient to prevent passive diffusion of the delivered at least one object; or
a substance that expands and covers the delivered at least one object.

7. The method of claim 1, wherein the retaining or depositing is carried out by a localized mechanical wave.

8. The method of claim 1, wherein the retaining or depositing is carried out by a sound emitter, mechanical wave emitter, energy conductor, sound source, and waveguide.

9. The method of claim 8, wherein the retaining or depositing is carried out by an ultrasound transducer or an ultrasound source.

10. The method of claim 1, wherein the retaining or depositing is carried out by an ultrasound, photo-acoustic or plasma source.

11. The method of claim 10, wherein the ultrasound or photo-acoustic source is configured to generate localized mechanical waves into a tissue based on high-intensity focused ultrasound (HIFU) or topological ultrasound.

12. The method of claim 10, wherein the ultrasound or photo-acoustic source is configured to generate localized mechanical waves into a tissue based on time reversal ultrasound or electromagnetic steering of a wave field or the at least one object.

13. The method of claim 1, wherein the retaining is carried out by an ultrasound or photo-acoustic source which is configured to cause formation of aggregates of the at least one drug by mechanical vibration.

14. The method of claim 1, wherein the at least one drug is encapsulated and the method further comprises:
prior to the depositing, releasing the at least one drug from encapsulation.

15. The method of claim 1, wherein the at least one drug is selected from the group consisting of analgesics, antagonists, anti-inflammatory agents, anthelmintics, antianginal agents, antiarrhythmic agents, antibiotics, anti-cholesterols, anticoagulants, anticonvulsants, antidepressants, antidiabetic agents, antiepileptics, a ntigonadotropins, antihistamines, antihypertensive agents, antimuscarinic agents, antimycobacterial agents, antineoplastic agents, antipsychotic agents, immunosuppressants, antithyroid agents, antiviral agents, antifungal agents, anxiolytic sedatives (hypnotics and neuroleptics), astringents, beta-adrenoceptor blocking agents, blood products and substitutes, anti-cancer agents, cardiacinotropic agents, contrast media, corticosterioids, cough suppressants (expectorants and mucolytics), diuretics, dopaminergics, haemostatics, immunosuppressive and immunoactive agents, lipid regulating agents, muscle relaxants, parasympathomimetics, parathyroid calcitonin and biphosphonates, prostaglandins, radiopharmaceuticals, sex hormones, steroids, anti-allergic agents, stimulants and anorexics, sympathomimetics, thyroid agents, vasidilators, neuron blocking agents, anticholinergic and cholinomimetic agents, antimuscarinic and muscarinic agents, vitamins, and xanthines.

16. The method of claim 1, wherein the object contains a precursor of the at least one drug and the method further comprises:
activating the at least one object by mechanical waves, electromagnetic waves or temperature.

17. The method of claim 1, wherein the object contains precursors of at least first and second drugs and the method further comprises:
activating the first drug, and
activating the second drug.
